(19) United States
(12) Patent Application Publication
(10) Pub. No.: US 2006/0201522 A1
(43) Pub. Date: Sep. 14, 2006

(54) Method for lowering blood glucose levels, method of treating diabetes, and method of prevention of diabetes

(76) Inventor: Yoshiaki Sato, Tokyo (JP)

Correspondence Address: MICHAEL BEDNAREK;SHAW PITTMAN LLP, 1650 TYSONS BOULEVARD, MCLEAN, VA, 22102, US

(21) Appl. No.: 11/075,875

(22) Filed: Mar. 10, 2005

(51) Int. Cl. A61B 19/00

(52) U.S. Cl. 128/898

(57) ABSTRACT

To provide a method of lowering blood glucose levels and a method of treating and preventing diabetes.

A belt-shaped compression strip 1 is fitted around a tightened area near the proximal end of an arm or a leg of a user and is fastened round the area. In this way, a compression pressure of on the order of 150 mmHg is applied to the tightened area to restrict blood flow through limbs receiving the compression pressure. This condition is held for about 15 minutes. During that time a user may keep rest or may do light exercises, which lowers a blood glucose level of the user. This provides treatment for diabetes for a diabetes patient and provides prevention of diabetes for a person who has a risk of developing diabetes.

### TECHNICAL FIELD

The present invention relates to a technique of lowering blood glucose levels, and to a technique of treating or preventing diabetes by using a method of lowering blood glucose levels.

### BACKGROUND ART

The present inventor has conducted studies for some time in order to work out a muscle training program for easy, safe, and effective muscle development, and put together the accomplishments into a patent application having Japanese Patent Application No. 5-313949, which has been granted as Japanese Patent No. 2670421. In addition, the present inventor filed a U.S. patent application, claiming priority to this earlier application, which has been granted as U.S. Pat. No. 6,149,618.

The muscle training program described in these applications is called a “Pressure Muscle Training Method” and has become popular in Japan because of its remarkable effects described below. Furthermore, domestic and foreign doctors and universities have conducted studies on it.

The present inventor has conducted further studies about pressure muscle training and filed some applications relating to tools and devices for use in the Pressure Muscle Training Method, which include: Japanese Patent Laid-open No. 7-144027, Japanese Patent Laid-open No. 10-85361, Japanese Patent Laid-open No. 10-85362, Japanese Patent Laid-open No. 2004-215858, Japanese Patent Laid-open No. 2004-313423, Japanese Patent Laid-open No. 2005-509, and Japanese Patent Laid-open No. 2005-6921.

The Pressure Muscle Training Method is a distinctive nonconventional one as described below. The Pressure Muscle Training Method is based on the following theoretical concept.

Muscles are composed of slow-twitch muscle fibers and fast-twitch muscle fibers. Slow-twitch muscle fibers are limited in their potential for growth. Accordingly, it is necessary to recruit fast-twitch muscle fibers of the slow-and fast-twitch muscle fibers in order to develop muscles. Recruitment of fast-twitch muscle fibers causes lactic acid buildup in the muscles, which triggers secretion of growth hormone from the pituitary. The growth hormone has effects of, for example, promoting muscle growth and shedding body fat. This means that recruitment of fast-twitch muscle fibers results in development of fast-twitch muscle fibers and, in turn, the entire muscles.

Slow-twitch muscle fibers and fast-twitch muscle fibers are different from each other in terms of the following. Slow-twitch muscle fibers use oxygen for energy and are recruited for low-intensity activities. Fast-twitch muscle fibers provide for activities even when no oxygen is present. They are recruited after the slow-twitch muscle fibers for highly intense activities. Therefore, it is necessary to cause the earlier recruited slow-twitch muscle fibers to be exhausted soon in order to recruit fast-twitch muscle fibers.

Conventional muscle training programs use exercises with, for example, a barbell to cause the slow-twitch muscle fibers to be exhausted first, and then to recruit the fast-twitch muscle fibers. This requires a significant amount of exercises, is time-consuming, and tends to increase the burden on muscles and joints.

On the other hand, restriction of blood flow to the limb(s) by applying a pressure to a predetermined tightened area or areas near the proximal ends of the limbs (and, in some cases, doing muscle exercises under such conditions) reduces the amount of oxygen carried to the muscles. The slow-twitch muscle fibers, which require oxygen for energy, are thus exhausted in a short period of time. Muscle exercises with blood-flow restriction by application of a pressure will result in recruitment of the fast-twitch muscle fibers without needing a large amount of exercises.

In addition, restriction of the blood flow by application of a pressure makes the lactic acid built up in the muscles less likely to be removed from the muscles. Thus, the muscle lactic acid level is more likely to rise and a much larger amount of growth hormone is secreted, as compared with the case where the blood flow is unrestricted.

Based on this theoretical concept, restriction of muscle blood flow can provide significant development of the muscles.

The Pressure Muscle Training Method according to the aforementioned Japanese and United States applications that have been patented, is premised on the theoretical concept of muscle development by the restriction of blood flow. More specifically, a predetermined compression pressure is applied to at least one of tightened areas of a user near the proximal ends of his or her limbs to put an appropriate stress attributed to blood flow decrease on the muscles, thereby causing muscle fatigue. Thus, effective muscle development is achieved.

The Pressure Muscle Training Method features muscle development without any exercises because it involves developing muscles by putting a stress attributed to blood flow decrease on the muscles. In addition, the Pressure Muscle Training Method can compensate for a total amount of stress that is placed on the muscles by putting a stress attributed to reduced blood flow on the muscles. When combined with some exercises, the method advantageously reduces an exercise-related stress as compared with conventional methods. This advantage brings about some effects: the possibility of incurring damages to the joints or muscles can be reduced and the period of training can be reduced, as a result of decrease in amount of muscle exercises.

### SUMMARY OF THE INVENTION

As apparent from the above, the Pressure Muscle Training Method was basically developed as a method for muscle development.

However, as a result of daily studies, the present inventor has found that the Pressure Muscle Training Method can lower a blood glucose level of a user who follows the Pressure Muscle Training Method.

The following inventions are based on the aforementioned findings that the present inventor has obtained.

The present invention is generally divided into a first invention and a second invention.

The first invention is a method of lowering blood glucose levels, comprising: applying a predetermined compression pressure to at least one of tightened areas of a user near the proximal ends of his or her limbs to restrict blood flow through the limbs receiving said compression pressure; and maintaining that condition for a predetermined period of time to lower a blood glucose level of the user.

This means that a blood glucose level of the user can be lowered when the user acts according to the conventional Pressure Muscle Training Method. Elevated blood-sugar levels can exert adverse effects on the body and cause progression of diabetes. The first invention has a good safety feature in that blood glucose levels can be lowered without administration of any drugs from outside the body. In addition, it is simpler than conventional procedures that need a device such as a syringe.

The following estimation holds for the reason why the blood glucose levels fall in response to restriction of the blood flow by application of a predetermined compression pressure to at least one of the tightened areas of a user near the proximal ends of the limbs. It has been found that restriction of the blood flow by application of a compression pressure to an area or areas of a user near the proximal ends of the limbs causes the pituitary of the user to secrete growth hormone in an amount that is unexpectedly greater than would ordinarily secrete. Secretion of growth hormone triggers secretion of insulin-like growth factor (IGF)-1 in the body of the user. IGF-1 can lower blood glucose levels. Therefore, the first invention can provide the aforementioned effect of lowering the blood glucose levels.

The secretion of growth hormone is transient, lasting a few hours or so, when the blood flow is restricted using the aforementioned method. On the other hand, the secretion of IGF-1 continues for generally two days. This means that the blood glucose level is kept low for generally two days when the restriction of blood flow is made using the aforementioned method. It should be noted that the growth hormone itself functions to lower the blood glucose levels, but secretion of the growth hormone is transient when the blood flow is restricted using the aforementioned method, so that this does not inhibit IGF-1 from lowering the blood glucose levels.

In the first invention, the user may keep rest while restricting the blood flow in said limbs receiving said compression pressure. Alternatively, the user may do exercises while restricting the blood flow in said limbs receiving said compression pressure. In addition, a combination of rest and exercises may be used.

When the user does exercises while restricting the blood flow in said limbs receiving said compression pressure, the exercises may cause muscles of said limbs receiving said compression pressure to flex. Alternatively, it may cause other muscles than those of the limbs receiving the compression pressure to flex. Although exercises of the limbs receiving the compression pressure are fundamental, the blood glucose levels may be lowered when the other muscles than those of the limbs are used for exercises.

The second invention is a method of treating or preventing diabetes, comprising: applying a predetermined compression pressure to at least one of tightened areas of a user near the proximal ends of his or her limbs to restrict blood flow through the limbs receiving said compression pressure; and maintaining that condition for a predetermined period of time to lower a blood glucose level of the user, thereby achieving treatment or prevention of diabetes.

It is widely known that a lowering of blood glucose levels is the most essential part of the treatment or prevention of diabetes. The second invention that can lower the blood glucose levels as in the first invention provides a method of treatment or prevention of diabetes.

Information announced from the International Diabetes Federation (IDF) in 2003 estimates that 16.02 million people in the United States suffer from diabetes and the total cost of diabetes treatment is around 66.71 billion dollars. In addition, it is said that the number of people who may be affected by diabetes, called the to-be diabetes patients, is several times larger than that of the diabetes patients.

As described above, the second invention has advantageous features in that it requires no administration of drugs, requires no such device as a syringe, and is enough when used every two days, as in the first invention. Therefore, the second invention is expected to be a great help for a huge number of diabetes patients or to-be diabetes patients as described above.

The user who follows the second invention may keep rest while restricting the blood flow in said limbs receiving said compression pressure. Alternatively, the user may do exercises while restricting the blood flow in said limbs receiving said compression pressure. This is the same as in the case of the first invention.

When the user who follows the second invention does exercises while restricting the blood flow in said limbs receiving said compression pressure, the exercises may cause muscles of said limbs receiving said compression pressure to flex. Alternatively, it may cause other muscles than those of the limbs receiving the compression pressure to flex. This is also the same as in the case of the first invention.

### BRIEF DESCRIPTION OF THE DRAWINGS

FIG. 1 is a perspective view illustrating how a compression strip used in a first embodiment is used;

FIG. 2 is a perspective view showing a modified version of a compression strip used in the first embodiment;

FIG. 3 is a view including a partially perspective showing the entire configuration of a compression device according to a second embodiment;

FIG. 4 is a cross-sectional view of a tight fitting band of a pneumatic belt included in the compression device shown in FIG. 3;

FIG. 5A is a view showing a clip that is used in combination with the pneumatic belt included in the compression device shown in FIG. 3, while FIG. 5B is a view illustrating how the clip is to be attached to the pneumatic belt;

FIG. 6 is a diagram showing a hardware configuration of a control mechanism contained in the compression device shown in FIG. 3;

FIG. 7A is a view showing an operation panel that is provided outside the compression pressure control unit included in the compression device shown in FIG. 3, while FIG. 7B is an enlarged view showing an example of an image displayed on a display unit on the operation panel; and

FIG. 8 is a perspective view illustrating how the pneumatic belt included in the compression device shown in FIG. 3 is used.

### BEST MODES FOR CARRYING OUT THE INVENTION

Preferred embodiments of the present invention are described now.

A method of lowering blood glucose levels as well as a method of treating and preventing diabetes according to this patent application may be achieved by using any one of implements and devices that allow a user to perform pressure muscle training. For example, a compression strip or a compression device disclosed in U.S. Pat. No. 6,149,618, Japanese Patent No. 2670421, Japanese Patent Laid-open Nos. 7-144027, 10-85361, 10-85362, 2004-215858, 2004-313423, 2005-509, and 2005-6921 may be used to practice the method of lowering blood glucose levels as well as the method of treating and preventing diabetes according to the present application.

The following description is for two of the implements and devices described in the aforementioned references, and for cases where they are used for the practice of a method of lowering blood glucose levels as well as a method of treating or preventing diabetes.

### First Embodiment

A compression strip 1 that is used in a method according to a first embodiment comprises a body unit 2 and a locking mechanism 3.

The body unit 2 has a shape of a belt made of an elastic material (e.g., rubber or a polyurethane resin). The length and the width of it may be appropriately determined depending on the anatomical shape of a user and whether the compression strip 1 is designed for an arm or a leg. The width of the body unit 2 may be on the order of 3-4 cm when designed for an arm and on the order of 5-6 cm when designed for a leg. The length of the body unit 2 is determined so that it is longer than the length around a predetermined site (tightened area) near the top of the limbs over which it is worn. The body unit 2 in this embodiment has a lining layer 4 made of a highly stretchable and highly water-absorbent material on the surface that directly contacts with the skin of the user.

The locking mechanism 3 is for maintaining the shape of the body unit 2 in order for the body unit 2 to rest around the tightened area. In this embodiment, the locking mechanism 3 comprises a first two-dimensional fastener 5a that is provided on one end of the body unit 2, a second two-dimensional fastener 5b that can engage the first two-dimensional fastener 5a in a removable manner, and a rectangular support ring 6 that is sewn on the body unit 2 at the other end thereof. The first two-dimensional fastener 5a and the second two-dimensional fastener 5b in this embodiment are both Velcro tapes. To wear the locking mechanism 3, the part of the body unit 2 on one end thereof where the first two-dimensional fastener 5a is provided is passed through the support ring 6 and is folded back towards the center of the body unit 2 at an appropriate position. Then, the first two-dimensional fastener 5a and the second two-dimensional fastener 5b are pressed together for adhesion. With the body unit 2 folded, a tension applied to the body unit 2 is adjusted to a desired degree and then the first two-dimensional fastener 5a is attached to the second two-dimensional fastener 5b for adhesion, thereby the pressure applied by the compression strip 1 around the tightened area to the tightened area can be kept at a desired pressure.

As the aforementioned compression strip 1, a compression belt (trade name: KINRYOKU UP-KUN) made by and available from SATO SPORTS PLAZA CO., LTD. may be used.

How the compression strip 1 is used is described.

First, the compression strip 1 is fitted to a predetermined tightened area. For this fitting, the locking mechanism 3 is used in the manner described above.

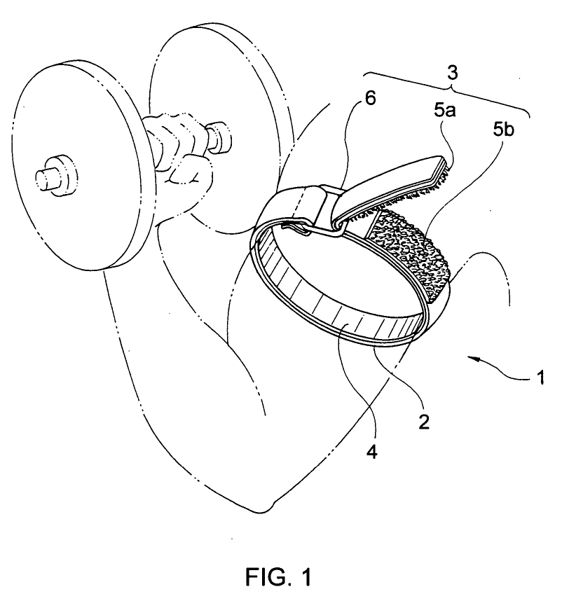

As shown in FIG. 1, the compression strip 1 in this embodiment is designed to be fitted around a tightened area on the arm. The tightened area is positioned between the deltoid and biceps muscles of the arm.

The compression strip 1 may be attached to tightened areas on both arms rather than on one arm. Alternatively, it may be attached to tightened area(s) of one or both legs. The tightened area on the leg is near the groin of the leg.

With this state, the compression strip 1 fastens round the tightened area at an appropriate pressure.

When the compression strip 1 is fitted around the tightened area, the tightened area receives an appropriate compression pressure applied by the compression strip 1. The appropriate compression pressure to be applied to the tightened area and a suitable time duration during which the compression pressure is applied to the tightened area depend on, for example, the sex, age, past history of exercises, and health condition. In general, the compression pressure may be on the order of 60 to 300 mmHg (in many cases, up to around 200 mmHg) when the tightened area is near the top of the arm) whereas it may be on the order of 90 to 350 mmHg (in many cases, up to around 280 mmHg) when the tightened area is near the groin. The time duration during which the pressure is applied is generally up to around 10 minutes when the tightened area is near the top of the arm whereas it is up to around 20 minutes when the tightened area is near the groin.

With this state, the blood flow through the limb(s) of the user that has the tightened area receiving the compression pressure is appropriately restricted.

With that state, the user may keep rest or do some exercises which may be light. When some exercises are used, the limbs having the tightened area receiving the compression pressure may be moved to flex the muscles of the limbs.

This lowers the blood glucose level of the user. Lowering of the blood glucose level begins to occur in several ten minutes after the beginning of application of the compression pressure to the tightened area. Although becoming weak gradually, the effect lasts for generally two days.

This achieves reduction of blood glucose levels in the user.

When the user is a diabetes patient, the aforementioned method provides a method of treating the diabetes. When the user belongs to a group of to-be diabetes who have a high risk of developing diabetes, then the aforementioned method provides a method of preventing the diabetes.

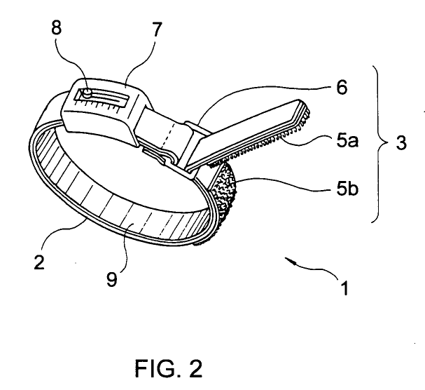

It should be noted that the compression strip 1 in the first embodiment may be modified as the one shown in FIG. 2. The compression strip 1 shown in FIG. 2 is different from the aforementioned compression strip 1 in that a display screen for compression pressure indication is provided on the body unit 2. The compression strip 1 also has the locking mechanism 3 that is similar in configuration to the locking mechanism 3 of the aforementioned compression strip 1.

The body unit 2 in the compression strip 1 may be made of a non-elastic material such as a fabric belt. It has a lining layer 9 made of a non-woven fabric, which is provided on the side that is to be contacted with the skin. In this example, a spring (not shown) is provided inside the display screen 7. Separated ends (not shown) of the body unit 2, which are separated away from each other at the site of the display screen 7, are connected with each other through the both ends of the spring. The amount of extension of the spring corresponding to the tightening force provided by the compression strip 1 is indicated as the displacement of a pointer 8 that is connected to the spring.

The display screen 7 may have other configuration than that described above. For example, it may electrically detect and display a compression pressure. In such a case, only the means for detecting the compression pressure is provided in the body unit and other means for displaying indication of a compression pressure according to the information about the compression pressure that has been received by wire or wireless from that means may be provided as a unit separate from the body unit 2. This allows reduction in weight of the body unit 2.

### Second Embodiment

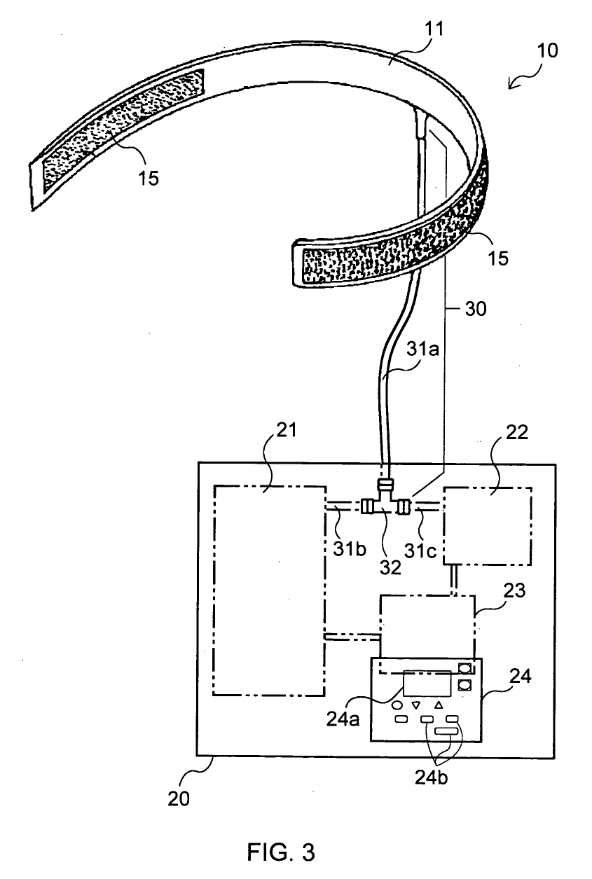

FIG. 3 is a view schematically showing a configuration of a compression device used in the method according to a second embodiment. This compression device includes a pneumatic belt 10 and a compression pressure control unit 20. They are connected to each other through a connection member 30.

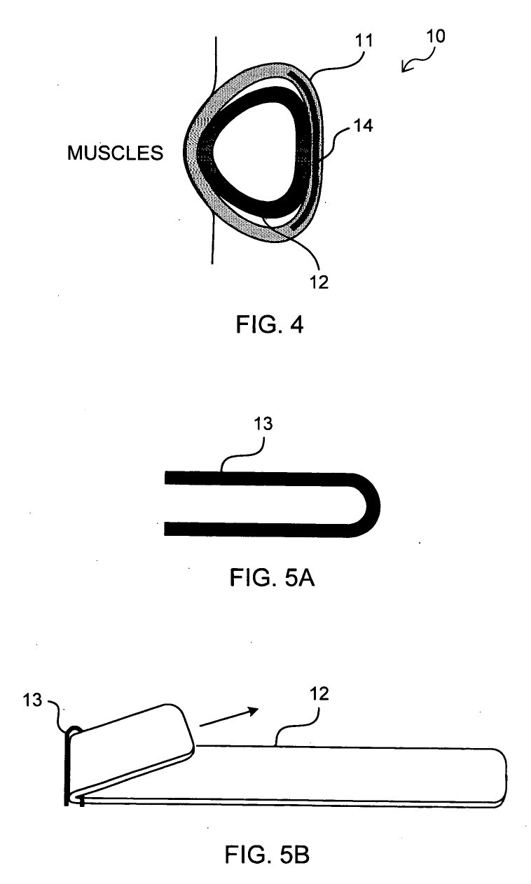

The pneumatic belt 10 in this embodiment is configured as shown in FIGS. 3 and 4. FIG. 3 is a cross-sectional view of the pneumatic belt 10 according to this embodiment.

The pneumatic belt 10 comprises a belt-shaped tight fitting band 11 that has a space inside it. The tight fitting band 11 is made up of two pieces of heavy fabric having a width of on the order of 5 cm. These pieces are stitched together along both longitudinal edges thereof into a loop. The fabric used on the side facing to muscles (inner side) of the tight fitting band 11 is made of stretch threads woven together to have a net-like appearance.

The tight fitting band 11 has a tube 12 therein. The tube 12 is made of a rubber that can withstand a pneumatic pressure of on the order of 300 mmHg.

The pneumatic belt 10 according to this embodiment further comprises a clip 13 as shown in FIG. 5A for delimiting the portion of the tube 12 into which air is introduced when attached to the tight fitting band 11 at a certain position along the length thereof.

The clip 13 has a shape of hairpin having two parallel straight segments and another segment connecting the one end of these two straight segments with each other. The length of the straight segment of the clip 13 is generally identical to the width of the tube 12. The distance between the straight segments is generally identical to the thickness of the tube 12. When used, the clip 13 is attached to the tube 12 in the widthwise direction of the tube in such a manner that the clip pinches the tube 12 as shown in FIG. 5B. This can delimit the range into which the air is introduced (the range inflated by the incoming air) of the tube 12 in the direction along the length of the tube.

The tight fitting band 11 has a limit piece 14 therein along the outer contour of the tube 12. The limit piece 14 is a plate-like member having a width of about 4 cm that is made of a polypropylene resin.

A two-dimensional fastener 15 is provided on the outer surface of the tight fitting band 11. The two-dimensional fastener 15 is for holding in place the tight fitting band 11 that is fitted around the tightened area of the user. The two-dimensional fastener 15 in this embodiment is a Velcro tape.

The connection member 30 in this embodiment is made up of connecting pipes 31a, 31b, and 31c and a T-shaped pipe 32 (FIG. 3). It should be noted that the connecting pipes 31b and 31c as well as the T-shaped pipe 32 are provided inside the compression pressure control unit 20. In addition, each of the connecting pipes 31a, 31b, and 31c is, but not limited to, a rubber tube and the T-shaped pipe 32 is made of a resin in this embodiment. The connecting pipe 31a can be attached to and removed from the T-shaped pipe 32.

It should be noted that while FIG. 3 illustrates only one pneumatic belt 10, the compression pressure control unit 20 in practice is connected to two pneumatic belts 10 designed for both right and left arms. The two pneumatic belts 10 designed for the right and left arms are identical to each other, so that only one of them is shown in FIG. 3 and illustration of the other is omitted.

It is apparent that the pneumatic belts 10 are not limited to those designed for the right and left arms. They may be two pneumatic belts designed for the right and left legs or a combination of four pneumatic belts designed for the two arms and two legs. If the Pressure Muscle Training Method is applied to two or more persons for some reasons, the pneumatic belt 10 of the number larger than that described above may be connected to the compression pressure control unit 20. This indicates that the number of the pneumatic belts 10 may be determined arbitrarily within the range of at least 1, when necessary.

The compression pressure control unit 20 is for controlling the pneumatic belt 10.

The compression pressure control unit 20 in this embodiment is configured with various parts and components provided inside and outside a casing.

The compression pressure control unit 20 has a pump 21, a pressure gauge 22, and a control mechanism 23 that are provided inside it as shown by broken lines in FIG. 3. The compression pressure control unit 20 also has an operation panel 24 that is provided outside it as shown by a solid line in FIG. 3. The operation panel 24 has a display unit 24a and an operation section 24b.

The control mechanism 23 in this embodiment may be electrically (but not limited thereto) connected to the pump 21, the pressure gauge 22 and the operation panel 24. The pump 21 and the pressure gauge 22 are connected to the tight fitting band 11 through the connecting pipes 31a, 31b, and 31c and the T-shaped pipe 32.

The pump 21 is for forcing the air to flow into the tube 12 within the tight fitting band 11 and sucking up the air from the tube 12. The pump 21 contains a motor that is not shown. By driving the motor, the pump can fill the tube 12 with the air or suck up the air from the tube 12. The pressure gauge 22 is for indirectly measuring the compression pressure applied to muscles by the tight fitting band 11 by means of measuring the air pressure within the tube 12 through the connection member 30. It should be noted that the compression pressure control unit 20 in this embodiment has pumps 21 for the right and left arms and pressure gauges 22 for the right and left arms. However, only one for each is illustrated in FIG. 3 because they are identical to their respective counterparts. The pump 21 and the pressure gauge 22 are paired, and are provided in the same number as the maximum number of the pneumatic belts 10 to be attached to the compression pressure control unit 20 in question.

The control mechanism 23 is for performing the below-described control including the control of the pump(s) 21. For example, the pump 21 is controlled by the control mechanism 23 when driven.

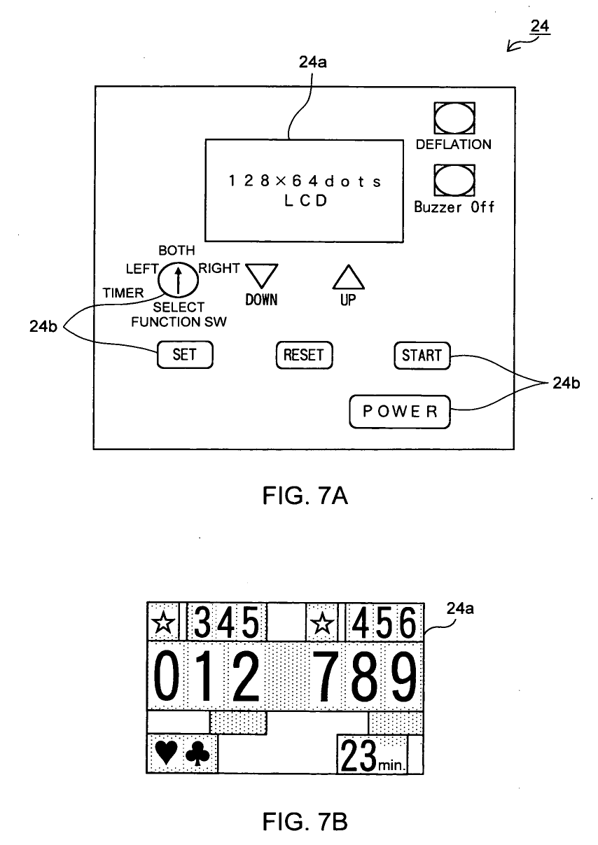

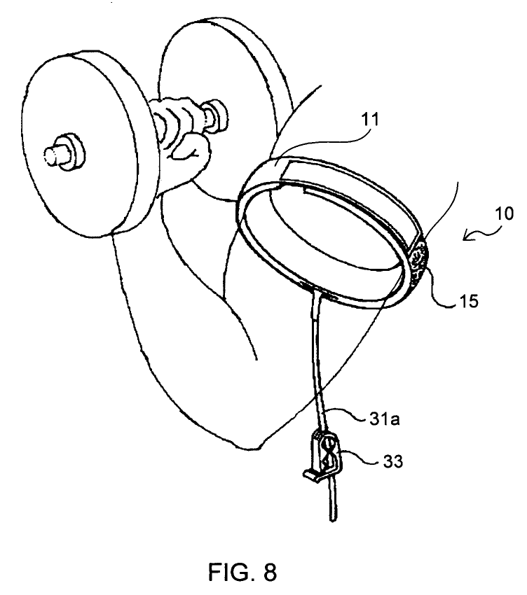

The operation panel 24 is shown in FIG. 7A in a simplified way.

As described above, the operation panel 24 comprises a display unit 24a and an operation section 24b as shown in FIG. 7A.

The display unit 24a in this embodiment is formed by using an LCD (liquid crystal display). It is apparent that the display device to be used for the display unit 24a is not limited thereto. Displayed on the display unit 24a are images including, for example, a value representing an air pressure within the pneumatic belt 10 and a numeric value indicating the elapsed time for application of a pressure. An example of the image displayed on the display unit 24a is shown in FIG. 7B.

The operation section 24b is provided with a switch that is used to turn the power supply on and off, a switch for setting the maximum value of the compression pressure and the maximum value of the duration during which a pressure is applied, and a switch for determining which one of a plurality of compression pressure profiles (programs recording how the compression pressure changes with time) that are previously recorded on the control mechanism 23 is to be called. It should be noted that the operation section 24b is also provided, in addition to the aforementioned switches, with a switch for deflating the tube 12 of the pneumatic belt 10 in case of, for example, emergency and a switch that is used to mute a buzzing sound.

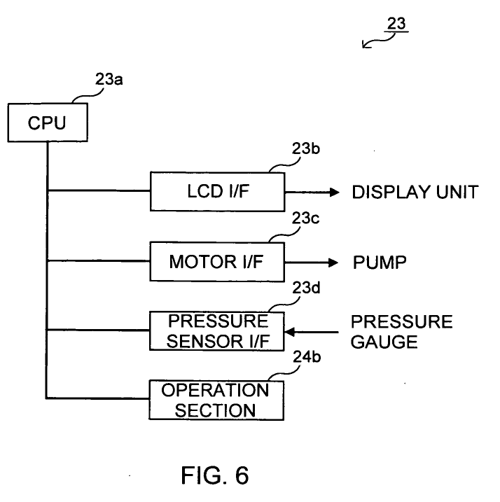

FIG. 6 shows a hardware configuration of the control mechanism 23. The control mechanism 23 comprises a CPU 23a, an LCD I/F 23b, amotor I/F 23c, a pressure sensor I/F 23d, and operation section 24b that are connected to each other via a bus. It should be noted that the bus is also connected to a ROM on which program(s) and data are stored which are used by the CPU 23a for performing necessary processes and to a RAM that provides a region for the processing of the program(s), which are not shown.

The CPU 23a is a processing unit that performs the processes carried out by the control mechanism 23 according to the program(s) and data read out of the aforementioned ROM. The CPU 23a is adapted either to control the LCD I/F 23b, the motor I/F 23c, the pressure sensor I/F 23d, and the operation section 24b or to receive data therefrom.

The LCD I/F 23b is an interface with the display unit 24a. The data generated by the CPU 23a for controlling the display unit 24a are supplied to the display unit 24a through this LCD I/F 23b.

The motor I/F 23c is an interface with a motor which is not shown and which is contained in the pump 21. The data generated by the CPU 23a for controlling the motor are supplied to the display unit 24a through this motor I/F 23c.

The pressure sensor I/F 23d is an interface with the pressure gauge 22. It accepts the data about the air pressure measured by the pressure gauge 22. These data are designed to be sent to the CPU 23a.

The CPU 23a is adapted to accept the data about the air pressure from the pressure sensor I/F 23d and control the aforementioned motor in the pump 21 in order to adjust the air pressure within the tube 12 to an appropriate level.

In doing so, the CPU 23a controls the pump 21 according to the data received from the operation section 24b in such a manner that a pressure is applied within a range of the maximum value of the compression pressure and the time duration during which a pressure is applied. In addition, when the user manipulates the operation section 24b to choose application of a pressure using a compression pressure profile recorded on the aforementioned ROM, then it controls the pump 21 based on the profile read out of the ROM according to the data received from the operation section 24b.

For example, the CPU 23a compares the data accepted from the pressure sensor I/F 23d with the data of a profile indicating an ideal compression pressure to be applied by the tight fitting band 11 to a wearer. If the difference between them exceeds a predetermined value, then the CPU generates such data in a real-time manner that allows the data accepted from the pressure sensor I/F 23d to approach the ideal compression pressure (or allows the pump 21 to be driven in such a manner that the air is sucked up from the tube 12 when the data accepted from the pressure sensor I/F 23d indicate a pressure that is higher than the ideal compression pressure, and allows the pump 21 to be driven in such a manner that the air is introduced into the tube 21 when the data accepted from the pressure sensor I/F 23d indicate a pressure that is lower than the ideal compression pressure).

It should be noted that, as the compression device as described above, a pneumatic training machine (trade name: KAATSU MASTER) manufactured by and available from SATO SPORTS PLAZA CO., LTD. may be used.

Next, with reference to FIG. 8, how the compression device is used is described.

In order to develop muscles using the compression device of the present invention, the tight fitting band 11 of the pneumatic belt 10 is fitted around an upper portion of the muscles desired to be developed. FIG. 8 shows a state in which the tight fitting band 11 is fitted around a tightened area near the proximal end of an arm. Fitting of the tight fitting band 11 around the tightened area is performed for both arms in this embodiment.

Prior to fitting the tight fitting band 11 around the arms, the clip 13 is attached to the tube 12 at an arbitrary position along the length of it in this embodiment. When the tube 12 is longer than the circumference of the tightened area of limbs, one end of the tube 12 is overlapped with the other end when the tight fitting band 11 is fitted around the arm from the other end of the tube 12. Such overlapped portions of the tube 12 produce a gap between the tube 12 and the muscles, which may cause a trouble in that a compression pressure to be applied to the muscles by the tight fitting band 11 becomes improper. Thus, the clip 13 is attached to the tube 12 at an arbitrary position along the length of it to restrict the length of the tube 12 that is filled with the air, thereby avoiding a problem as described above. In other words, the clip 13 is attached as shown in FIG. 3B in this embodiment. Thus, the air can flow only into the portion of the tube 12 that corresponds to the circumferential length of the portion of the arm on which the tight fitting band 11 is worn. The air has no passage beyond that point.

To this end, the tube 12 is allowed to be removed from the tight fitting band 11.

Next, the tight fitting band 11 is fixed to the two-dimensional fastener 15 in such a manner that the diameter of a loop formed by the tight fitting band 11 does not change.

In this case, it is made carefully without producing any gap between the arm and the tight fitting band 11 because any gap between the arm and the tight fitting band 11 breaks the corresponding relationship between the air pressure to the tube 12 within the tight fitting band 11 and the compression pressure generated by the tight fitting band 11.

In this state, driving of the compression pressure control unit 20 causes the tight fitting band 11 to tighten the arm.

More specifically, when the compression pressure control unit 20 is driven, the air is supplied from the pump 21 controlled by the control mechanism 23 to the tube 12. At that time, the pump 21 is appropriately controlled according to the data generated by the control mechanism 23 for driving the pump 21 depending on the air pressure in the tube 12 that is monitored by the pressure gauge 22, as described above. This maintains the air pressure in the tube 12 at a suitable level, and the compression pressure applied by the tight fitting band 11 to the muscles is also maintained at a suitable level.

The segment of the air-receiving tube 12 of which boundary is defined by the clip 13 and which includes a portion connected to the connecting pipe 31a is inflated. In this case, the tube 12 is being inflated inwardly against the muscles and is also being inflated outwardly. The outside of the tube 12 is, however, limited by the limit piece 14. Thus, the direction of inflation of the tube 12 is limited to the inward direction against the muscles, as shown in FIG. 4. This results in an appropriate compression pressure being applied by the tight fitting band 11 to a predetermined tightened site on the limbs as the tube 12 is inflated.

In this state, the wearer may keep rest for a while or put a stress on the muscles by doing some exercises of the arm including the tightened area to which a pressure is being applied.

When the muscle exercises are used, the connecting pipe 31a, of which proximal end is connected to the compression pressure control unit 20, may get in the way. In such a case, a part of the connecting pipe 31a may be closed by, for example, pinching it with a stopper 33 such as a clip to avoid leakage of the air from the tube 12 of the tight fitting band 11, and then the connecting pipe 31a and the T-shaped pipe 32 may be separated. The aforementioned FIG. 7 shows a state where the stopper 33 is used to close the connecting pipe 31a. It should be noted that, as a result of this, the compression pressure control unit 20 becomes unavailable for controlling the air pressure in the tube 12, so that the compression pressure applied to the tightened area is fixed to the compression pressure at the time when the connecting pipe 31a is closed by the stopper 33.

By following the aforementioned methods, the blood glucose levels of the user can be lowered.

In addition, when the user is a diabetes patient, the aforementioned method provides a method of treating the diabetes. When the user belongs to a group of to-be diabetes who have a high risk of developing diabetes, then the aforementioned method provides a method of preventing the diabetes.

While the aforementioned method may be used every day (may be used several times a day), the effect of lowering the blood glucose levels can be obtained when it is used generally every two days.

What is claimed is:

1. A method of lowering blood glucose levels, comprising:
applying a predetermined compression pressure to at least one of tightened areas of a user near the proximal ends of his or her limbs to restrict blood flow through the limbs receiving said compression pressure; and
maintaining that condition for a predetermined period of time to lower a blood glucose level of the user.

2. The method of lowering blood glucose levels as claimed in claim 1, wherein the user keeps rest while restricting the blood flow in said limbs receiving said compression pressure.

3. The method of lowering blood glucose levels as claimed in claim 1, wherein the user does exercises while restricting the blood flow in said limbs receiving said compression pressure.

4. The method of lowering blood glucose levels as claimed in claim 3, wherein said exercises cause muscles of said limbs receiving said compression pressure to flex.

5. A method of treating or preventing diabetes, comprising:
applying a predetermined compression pressure to at least one of tightened areas of a user near the proximal ends of his or her limbs to restrict blood flow through the limbs receiving said compression pressure; and
maintaining that condition for a predetermined period of time to lower a blood glucose level of the user,
thereby achieving treatment or prevention of diabetes.

6. The method of treating or preventing diabetes as claimed in claim 5, wherein the user keeps rest while restricting the blood flow in said limbs receiving said compression pressure.

7. The method of treating or preventing diabetes as claimed in claim 5, wherein the user does exercises while restricting the blood flow in said limbs receiving said compression pressure.

8. The method of treating or preventing diabetes as claimed in claim 7, wherein said exercises cause muscles of said limbs receiving said compression pressure to flex.
